(19) United States
(12) Patent Application Publication
(10) Pub. No.: US 2007/0238921 A1
(43) Pub. Date: Oct. 11, 2007

(54) Capsule imaging device using fluorescent light

(76) Inventor: John Paul Weirich, Palo Alto, CA (US)

Correspondence Address: John Weirich, 524 Kendall Ave. #3, Palo Alto, CA, 94306, US

(21) Appl. No.: 11/248,967

(22) Filed: Oct. 12, 2005

Related U.S. Application Data
(60) Provisional application No. 60/624,003, filed on Nov. 1, 2004.

(51) Int. Cl. A61B 1/04

(52) U.S. Cl. 600/109

(57) ABSTRACT

A pill shaped device for in vivo imaging of tissues of the digestive system. The capsule imaging device gathers images of tissues of the gastro-intestinal tract that have been tagged by fluorescent dye. The light source of the device causes the fluorescent dye tagging the tissues to emit light images that are recorded by the sensor in the device and then transmitted to a remote system for processing.

### CROSS-REFERENCE TO RELATED APPLICATIONS

This application is related to and claims priority from U.S. Provisional Patent Application No. 60/624,003, titled “Fluorescent Dye Capsule Imaging Device”, filed on Nov. 1, 2004, which is hereby incorporated herein by reference for all purposes.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The present invention relates to generally to in vivo imaging devices and to in vivo imaging of tissues of the gastro-intestinal system tagged by fluorescent dyes.

A familiar device for imaging the gastro-intestinal (GI) tract is the Pillcam™ Capsule Endoscopy System sold by Given Imaging. This product produces a visible light video image of the interior wall of the GI tract as the capsule camera device is pushed through it. This device is described in U.S. Pat. No. 5,604,531.

Research teams such as those at the University at Buffalo's Institute for Lasers, Photonics and Biophotonics, the Quantum Dot Corporation, and Evident Technologies Inc., have developed quantum dot dye technology that is used to tag targeted cells with fluorescent quantum dot markers. Quantum dots emit visible or infrared light when they are stimulated with ultraviolet (UV), blue, or laser light, depending on the construction of the quantum dot.

When light at an activating frequency is absorbed by a quantum dot, the quantum dot reacts to this stimulus by emitting light in the visible or infrared frequency range, according to characteristics of the quantum dot.

Other fluorescent dyes, which are not quantum dots, are used in labs worldwide in imaging applications. These dyes are designed to attach themselves to targeted cells. These dyes are stimulated to fluoresce visible light by shining UV, blue, or laser light on them, similar to the technique used for stimulating quantum dots.

Quantum dot and other dye compounds can be produced and used to tag cancer tissues that grow in the GI tract. This has been done by Quantum Dot Corporation and by researchers at the Georgia Institute of Technology, among others.

A problem with Given Imaging's Pillcam™ Capsule Endoscopy System is that it does not cause fluorescent dye or quantum dot tags to emit light as summarized above. This is because the Pillcam does not use an ultraviolet, blue, or laser light source.

It is advantageous to have a system that uses fluorescent means, like quantum dots or other dyes, to tag targeted tissues in the GI tract, which can then be imaged by shining light on these tissues at a stimulating frequency and capturing the resulting fluoresced emitted images, by means of a camera sensor device and associated components.

### BRIEF SUMMARY OF THE INVENTION

It is an object of the present invention to provide an imaging sensor device for the gastro-intestinal (GI) system. In a preferred embodiment, this imaging sensor device is comprised of an ultraviolet light (UV) emitting diode (LED), a charge-coupled device (CCD) camera sensor, a battery power source, and a transmitter. These electronic components are enclosed in a pill sized indigestible shell that allows the electromagnetic light waves emitted and received by the device to pass through.

In accordance with an alternative embodiment of the present invention, a laser diode is substituted for the UV LED.

In accordance with another alternative embodiment, a blue light LED is substituted for the UV LED.

In accordance with an additional alternative embodiment, a CMOS camera sensor is substituted for the CCD camera sensor.

In accordance with another alternative embodiment, a transceiver component is substituted for the transmitter component.

Further, other alternative embodiments of the present invention could combine the components listed above into different functional combinations. For example, a desired imaging device could be comprised of a blue light LED, a CMOS camera, a transceiver, and battery, enclosed within the capsule shell. As another example, the desired imaging device could be comprised of both an UV LED and a laser diode, a CCD camera, a transmitter, and a battery, within the capsule. In the future, a miniature fuel cell could be substituted for the battery power source, if such fuel cells are developed.

The imaging device described in the embodiments above, could be used to illuminate and capture images of fluorescing quantum dots, or other dyes, which have been used to tag tissues in the GI tract. The capsule device illuminates and thereby stimulates the dye tags in the GI tract causing them to fluoresce, and takes pictures of these fluorescing tags as they lay embedded in the tissues of the GI tract, then transmits these picture images of the fluorescing tissues of the GI tract to the reception system. The reception system being similar to that described in U.S. Pat. No. 5,604,531.

Several companies sell CMOS sensor camera components, including Rohm, OmniVision, Mitsubishi, and Kodak. Companies selling CCD sensor camera components include Hamamatsu, Spectra-Physics, and Sony. Rockwell and Foveon sell infrared CMOS sensors.

Laser diodes and ultraviolet LEDs are available from companies such as Roithner Lasertechnik, Lumex, Chi Wing, and Laser Diode Inc. White light LEDs are generally available in the market, as are blue light LEDs.

In the preferred embodiment of the present invention, an UV LED is activated by a battery power source causing the LED to emit UV waves through the capsule shell which is transparent to these waves.

The UV waves are absorbed by the quantum dot tags that are located in tissues of the wall of the GI tract. These quantum dot tag markers are typically associated with targeted diseased tissue, for example cancer tissue.

When the UV waves are absorbed by the quantum dots, the quantum dots fluoresce visible light which is recorded by the CCD camera sensor also located within the capsule shell. The CCD camera sensor sends these images to the transmitter for broadcast to the receiver located outside the body of the subject.

As stated above, an alternative embodiment substitutes a laser diode for the UV LED. In this case, laser light is emitted from the capsule to stimulate the quantum dots, or other type of fluorescent dye, tagging the targeted tissue. Similarly, in another embodiment, a blue light LED is substituted for the UV LED. The blue light causes the tags to fluoresce.

If the fluorescent dye tags are designed to glow in the infrared frequency band when stimulated by the light from the capsule device, then a CMOS infrared sensor would be used as the image receiving component in the capsule.

In another alternative embodiment, a CMOS camera sensor is substituted for the CCD camera sensor to receive the light fluoresced by the dye.

According to an additional alternative embodiment, both a white light LED is used in combination with a laser diode to illuminate the GI tract tissues. In this embodiment, the camera sensor component would record both the light reflected from the GI tissues resulting from illumination with the white light LED, and the visible light fluoresced by the dye as a result of being stimulated by the laser light.

Other alternative embodiments could substitute a green or red or yellow, or other visible light LEDs, for the white light LED.

Many different combinations of components could be joined together into a useful capsule imaging device, and the alternatives listed are not to be considered as limiting or exhaustive in any sense.

### BRIEF DESCRIPTION OF THE SEVERAL VIEWS OF THE DRAWINGS

FIG. 1 is a cut-away side view of the components of the capsule imaging device.

FIG. 2 is a side view of the front part of the capsule imaging device.

### DETAILED DESCRIPTION OF THE INVENTION

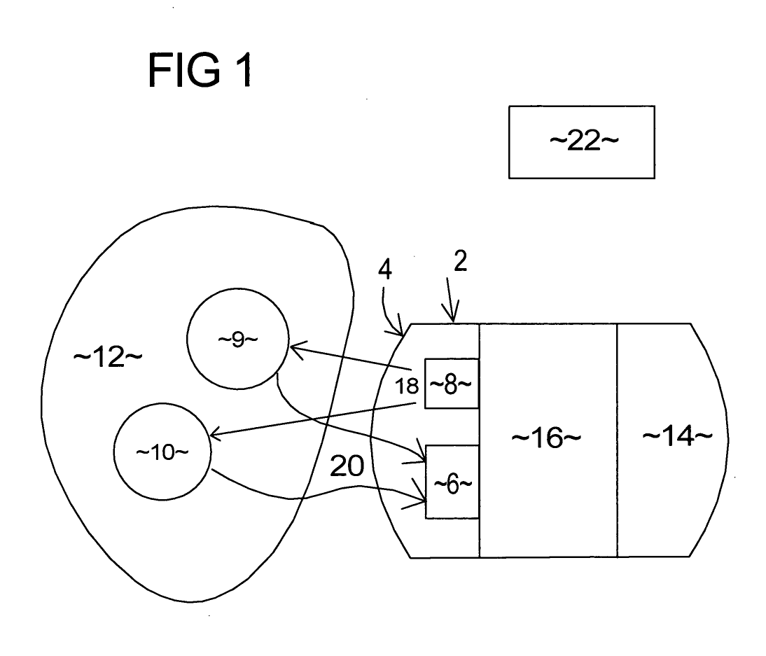

FIG. 1 illustrates the components of the preferred embodiment of the present invention, here the shell 2 of the capsule houses its parts. The UV LED 8 is located to direct UV waves 18 through the transparent shell window area 4. Some of these UV waves 18 strike quantum dots 9, 10 tagging cancer cells 12 in the tissues of the GI tract.

A CCD camera sensor 6 is located to receive visible light waves 20 fluoresced from the quantum dot tags 9, 10 in response to their having been stimulated by the UV waves 18.

When the CCD camera sensor 6 detects these light waves 20, the sensor 6 relays this imaging information to the transmitter 16 for broadcast to the receiver system 22 located outside the body of the subject. The receiver system 22 could be of the type described in U.S. Pat. No. 5,604,531. The battery 14 provides electrical power for all these operations. All of these electrical components are operatively connected by electrical circuitry.

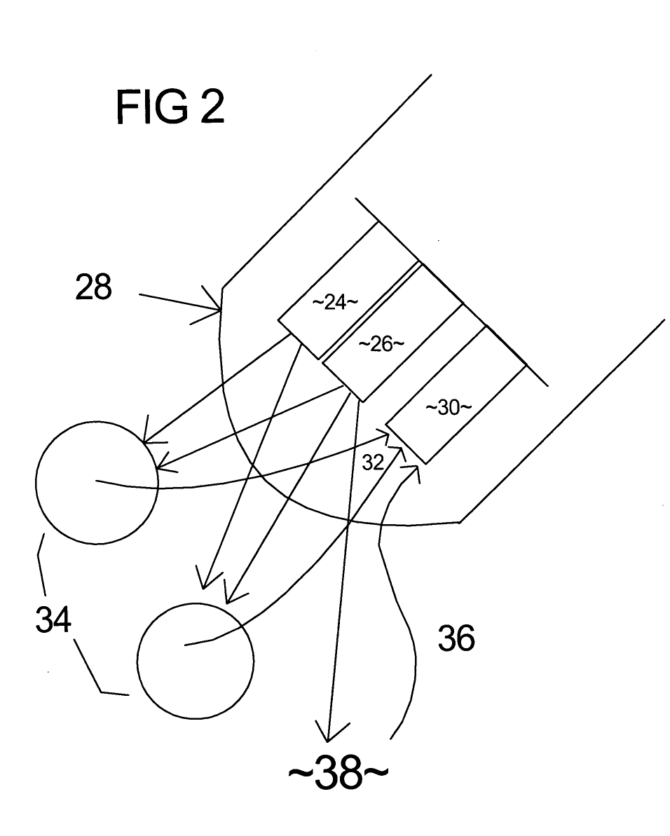

FIG 2 shows a side view of the front section of the capsule imaging device. Here two illuminating components operate together. The UV LED 24 and white light LED 26 are directed to project UV and white light, respectively, through the transparent capsule shell 28. The CCD camera sensor 30 is located to detect the visible light waves 32 emitted from the quantum dots 34 and visible light 36 reflected from the GI tract tissues 38.

Alternative embodiments of the present invention can be envisioned and realized. Some examples are: the UV LED 8 FIG. 1 could be replaced with a laser diode; the CCD camera sensor 6 FIG. 1 or 30 FIG. 2 could be replaced with a CMOS camera sensor; the white light LED 26 FIG. 2 could be replaced with a green, yellow, blue, red, orange, or other colored visible light or infrared light LED; or the transmitter 16 FIG. 1 could be replaced with a transceiver. Any substitutions like these mentioned, or similar component substitutions, could be done individually or severally, depending on desired device characteristic goals.

The use of a laser diode or a UV LED, either individually or in combination with other light sources, to illuminate targeted tissue is a distinguishing characteristic of the present invention from the prior art.

Although specific embodiments of the present invention have been described in detail above, it will be recognized by those skilled in the art that the present invention is not limited by these descriptions. In fact, variations can be made to the embodiments described without varying from the spirit of the present invention. Rather the scope of the present invention is defined only by the following claims:

I claim:

1. A capsule imaging system comprising:
a capsule shell to enclose the components; and
a ultraviolet light source; and
a camera sensor; and
a battery; and
a transmitter; and
fluorescent dye means for tagging tissue.

2. A system according to claim 1, and wherein said ultraviolet light source is a laser light source.

3. A system according to claim 1, and wherein said ultraviolet light source is combined with one or more other light sources.

4. A system according to claim 2, and wherein said laser light source is combined with one or more other light sources.

5. A system according to claim 1, 2, 3, or 4, and wherein said fluorescent dye means is quantum dot dye.

6. A system according to claim 1, 2, 3, or 4, and wherein said fluorescent dye means is non-quantum dot chemical dye.

7. A system according to claim 1, 2, 3, 4, 5, or 6, and wherein said transmitter is a transceiver.

8. A capsule imaging system comprising:
a capsule shell to enclose the components; and
a ultraviolet light source; and
a camera sensor; and
a battery; and
a transmitter.

9. A system according to claim 8, and wherein said ultraviolet light source is a laser light source.

10. A system according to claim 8, and wherein said ultraviolet light source is combined with one or more other light sources.

11. A system according to claim 9, and wherein said laser light source is combined with one or more other light sources.

12. A system according to claim 8, 9, 10, or 11, and wherein said transmitter is a transceiver.
